SEQUENCE LISTING (3 sequences)

SEQ ID NO: 1 - protein, 34 aa, Artificial Sequence
  sSP-Css-ion lock
GIPSSPVHLKRLLIVVVVVELIVKVIVGALLMGL

SEQ ID NO: 2 - protein, 34 aa, Artificial Sequence
  sSP-Cff-leuc
GIPFFPVHLKRLKLLLLLLLLILLLILGALLMGL

SEQ ID NO: 3 - protein, 34 aa, Artificial Sequence
  sSP-Cff-ion lock
GIPFFPVHLKRLLIVVVVVELIVKVIVGALLMGL

(19) United States
(12) Patent Application Publication
(10) Pub. No.: US 2024/0091319 A1
(43) Pub. Date: Mar. 21, 2024

(54) DUAL-ADMINISTRATION METHODS FOR TREATING RESPIRATORY DISTRESS

(71) Applicant: THE TRUSTEES OF THE STEVENS INSTITUTE OF TECHNOLOGY, Hoboken, NJ (US)

(72) Inventor: Carrie E. PERLMAN, New York, NY (US)

(73) Assignee: THE TRUSTEES OF THE STEVENS INSTITUTE OF TECHNOLOGY, Hoboken, NJ (US)

(21) Appl. No.: 18/272,549

(22) PCT Filed: Jan. 18, 2022
(86) PCT No.: PCT/US2022/012832

§ 371 Date: Jul. 14, 2023

Related U.S. Application Data
(60) Provisional application No. 63/138,393, filed on Jan. 15, 2021.

(51) Int. Cl. A61K 38/38; A61K 38/17

(52) U.S. Cl. CPC A61K 38/38; A61K 38/395; A61K 9/0019

(57) ABSTRACT

A method of treating a patient having edematous lungs includes: administering a first surface tension-lowering component to the patient via the airways and administering a second surface tension-lowering component to the patient via the vasculature. The combined and complementary treatments may lower or normalize alveolar surface tension with improved efficiency and on a faster timescale, thereby reducing patient injury and mortality (FIG. 1).

### CROSS-REFERENCE TO RELATED APPLICATIONS

This application claims priority to and the benefit of U.S. Provisional Patent Application Ser. No. 63/138,393 filed Jan. 15, 2021, the entire disclosure of which is incorporated herein by reference.

### STATEMENT REGARDING FEDERALLY SPONSORED RESEARCH

This invention was made with government support under Grant Number RO1 HL113577 awarded by the NIH. The U.S. government has certain rights in the invention.

### BACKGROUND

In treatment of patients with neonatal respiratory distress syndrome (NRDS), acute respiratory distress syndrome (ARDS), or cardiogenic pulmonary edema (CPE), liquid floods the airspace of the lungs, and ventilation due to spontaneous breathing or mechanical ventilation (which is often used to support gas exchange) can cause ventilation-induced lung injury (VILI), which contributes to high mortality. The risk of VILI is proportional to the interfacial surface tension (T) in the alveoli, which is elevated in NRDS and ARDS. Thus, adjuvant therapies that reduce T may be associated with improved patient outcomes. Exogenous surfactant therapy, in which a surfactant is administered via the trachea, has been previously shown to be effective in NRDS but not ARDS. Improved therapeutic methods of lowering T are desired.

### SUMMARY

Embodiments of the present disclosure pertain to treatment methods for patients who have edematous or liquid-flooded lungs. The combined and complementary treatment methods disclosed herein may lower or normalize alveolar surface tension with improved efficiency and on a faster timescale, thereby reducing patient injury and mortality by administering a first surface tension-lowering component via the patient's airways in conjunction with administering a second surface tension-lowering component via the patient's vasculature.

In some embodiments, administering the first surface tension-lowering component involves tracheal instillation of the first surface tension-lowering component.

In some embodiments, administering the first surface tension-lowering component involves aerosolizing (e.g., nebulizing) the first surface tension-lowering component and continuously applying the aerosol to the patient's airways during a time period determined to deliver an appropriate amount of the surface tension-lowering component to the patient's airways.

In some embodiments, the first surface tension-lowering component can be administered via lavaging the patient's airways.

In some embodiments, administering the first surface tension-lowering component and administering the second surface tension-lowering component are carried out simultaneously. Alternatively, administering the first surface tension-lowering component can be carried out prior to administering the second surface tension-lowering component. Conversely, administering the first surface tension-lowering component can be carried out after administering the second surface tension-lowering component.

In some embodiments, administering the first surface tension-lowering component and/or administering the second surface tension-lowering component may be repeated at least once.

In some embodiments, administering the second surface tension-lowering component is carried out via a continuous infusion or drip, a single bolus injection and/or multiple injections.

In some embodiments, when administration of the first and/or second surface tension-lowering component is repeated more than once or is continuous, the time periods during which the two surface tension-lowering components are administered might be coincident, might partially overlap or might not overlap. During administration of the first surface tension-lowering component in aerosolized form, the patient's lungs may or may not be mechanically ventilated in accordance with embodiments of the disclosure.

In some embodiments, the first surface tension-lowering component comprises at least one surfactant.

In some embodiments, the first surface tension-lowering component comprises at least one surfactant, which may be diluted to less than 100 wt % of a commercial formulation.

In some embodiments, the first surface tension-lowering component comprises a negatively charged solute, such as albumin, fibrinogen, or negatively charged dextran. A negatively charged solute, such as albumin, fibrinogen, or negatively charged dextran can also be delivered via the patient's vasculature.

In some embodiments, the first surface tension-lowering component comprises a surfactant protein C (SP-C), in addition to, or in the absence of, a negatively charged solute (e.g., albumin, fibrinogen, or negatively charged dextran).

In some embodiments, the first surface tension-lowering component comprises at least one rhodamine dye such as sulfofhodamine B (SRB) and/or rhodamine WT (RWT), which may, for instance, have a concentration selected to achieve a concentration of rhodamine dye in the patient's alveolar liquid of about 1 nM to about 1,000 nM. With or without the presence of SP-C and/or at least one rhodamine dye, a negatively charged solute, such as albumin, fibrinogen, or negatively charged dextran can also be delivered via the patient's vasculature.

In some embodiments, the second surface tension-lowering component comprises at least one rhodamine dye such as SRB and/or RWT, which may, for instance, have a concentration selected to achieve a concentration of rhodamine dye in the patient's alveolar liquid of about 1 nM to about 1,000 nM and/or in the patient's plasma of about 1 nM to about 10,000 nM when administered.

In some embodiments, the second surface tension-lowering component comprises at least one rhodamine dye and/or albumin.

In some embodiments, the second surface tension-lowering component comprises surfactant protein C and/or a negatively charged solute.

With or without the presence of SP-C and/or at least one rhodamine dye, a negatively charged solute, such as albumin, fibrinogen, or negatively charged dextran can also be delivered via the patient's airway.

### BRIEF DESCRIPTION OF THE FIGURES

These and other aspects of the present disclosure will be better understood by reference to the following detailed description when considered in conjunction with the accompanying drawings, in which:

FIG. 1 is a chart which compares average alveolar surface tension in isolated rat lungs treated in vitro with various solutions. Treatment details disclosed in FIG. 1 and its chart, including the compositions and concentrations of any solutions administered by direct alveolar injection, are shown below the x-axis of the chart;

FIG. 2A shows Injury Score for Flooding Solutions Containing Specific Surfactant Components in the Presence of Albumin. Solutes are at about the same concentrations as present in 1% SURVANTA® solution. The surfactant protein B (SP-B) and SP-C used are isolated from pulmonary alveolar proteinosis patients; the SP-C, thus, is likely a mixture of fully-, partially- and non-palmitoylated peptide. Two forms of synthetic SP-C are used. One is sSP-Cff-leuc. The other is sSP-Cff-ion lock-B. Only SP-C and sSP-C lower injury score, thus lower surface tension. Base solution for all groups is normal saline with 5% albumin. Due to pre-dissolution of certain solutes, at high concentration, in non-aqueous solvents, the final dipalmitoylphosphatidylcholine (DPPC) solution contains 2% methanol; the final SP-B and SP-C solutions contain 1.6% chloroform and 0.8% methanol; and the final sSP-Cff-leuc solution contains 1.3% chloroform and 1.3% ethanol. The sSP-Css-ion lock-B is dissolved directly in 5% albumin solution in normal saline, without additional solvents. n=1 or 2/group. From these results it appears that albumin facilitation of dilute SURVANTA® solution is attributable to album in-SP-C interaction;

FIG. 2B. shows Injury Score for Flooding Solutions Containing SP-C, With and Without Albumin and DPPC. In normal saline without albumin, SP-C fom pulmonary alveolar proteinosis patients loses its ability to lower injury score, thus lower surface tension. Inclusion of DPPC does not restore the ability of SP-C to lower injury score, thus surface tension, in the absence of albumin. Base solution for all groups is normal saline. Due to pre-dissolution of certain solutes, at high concentration, in non-aqueous solvents, the final SP-C solutions, with or without albumin, contain 1.6% chloroform and 0.8% methanol; and the final SP-C plus DPPC solution contains 1.6% chloroform and 2.8% methanol. n=1 or 2/group. From these results it appears that isolated SP-C is not surface active on its own but is surface active when facilitated by albumin;

FIG. 3 shows Opening Pressure of the Immature Fetal Rat Lung Following Solution Instillation in the Trachea. Solution (4-5 μl), with solutes as specified, is instilled in the trachea of the fluid-filled immature (embryonic day 18 or 19) fetal rat lung. The pressure required to inflate the fetal rat lung for the first time is proportional to surface tension. Base solution is normal saline excepting that base solution is Ringer's solution for 1% SURVANTA® plus 5% albumin. The solution of 0.0005% SP-C plus 5% albumin additionally includes 1.6% chloroform and 0.8% methanol. The sSP-Cff-ion lock used is not biotinylated and is dissolved directly in normal saline without additional solvents. n=1 or 2/group. From these results it appears that 1% SURVANTA or isolated or synthetic SP-C, in combination with 5% albumin, is at least as effective as 100% SURVANTA in the absence of albumin; and

FIG. 4 shows Surface Tension for Solutions In Vitro. Surface tension of normal saline drops (3 μl) containing 31 μM fluorescein and additional solutes as specified. The SP-C solution additionally contains 1.6% chloroform and 0.8% methanol. The fourth, sixth and seventh bars show that, in the presence of 5% albumin, surface tension decreases with increasing SURVANTA® concentration. The third, fourth and fifth bars demonstrate that there is an optimal albumin concentration of ^{˜}5% for the facilitation of 1% SURVANTA®. n=2 or 3/group.

### DETAILED DESCRIPTION

Embodiments of the present disclosure relate to methods for minimizing or reducing ventilation injury to an edematous lung or liquid-flooded lung (e.g. neonatal lung from which liquid has been incompletely expelled), for example in a patient subjected to mechanical ventilation. For example, embodiments of the present disclosure provide a method of lowering the surface tension (T) of liquid in alveoli of the edematous or liquid-flooded lung by complementary (e.g., combined or simultaneous) administration of a surface tension-lowering component via the vascular system, and a surface tension-lowering component via the airways. In some embodiments, the surface tension-lowering component administered via the vascular system may include a rhodamine dye.

### Lung Physiology

During inhalation, air travels from the nose or mouth to the lungs via the airways. The trachea splits off to the two lungs via the main bronchi, after which the airways gradually branch into increasingly smaller structures. Following the bronchi are the bronchioles, the alveolar ducts and sacs, and finally the alveoli. The alveoli are the terminal airspaces of the lungs, where gas exchange (e.g., blood oxygenation and release of carbon dioxide) takes place. In normally functioning lungs, the air pressure is equal between two adjacent alveoli. Thus, as equal pressures are applied to each side of any wall (septum) between adjacent air-filled alveoli, the septum is substantially planar in shape. Pulmonary capillaries are located within the alveolar septa. The tissue and liquid between the alveolar air and the blood within those capillaries constitute the alveolar capillary barrier, across which gas exchange occurs.

The surface of an alveolus is lined with type I and II alveolar epithelial cells, on top of which there is a thin liquid lining layer. Thus, there is an air-liquid interface in the airspace of the lungs that has an associated surface tension. Alveolar type II epithelial cells naturally release a lung surfactant that adsorbs to the interface and maintains a low (e.g., physiologically appropriate) alveolar surface tension. The low surface tension achieved by the surfactant reduces the pressure required to keep the lungs inflated, and thereby reduces the effort required for breathing.

Endogenous lung surfactant is a mixture of phospholipids, the most abundant of which is dipalmitoylphosphatidylcholine (DPPC); neutral lipids; and four surfactant-associated proteins, surfactant protein (SP)-A, SP-B, SP-C, and SP-D. Surfactant proteins B and C, which are hydrophobic, facilitate surfactant lipid adsorption.

In various disease or injury states, including those described herein, the lung surfactant is impaired or is insufficient and does not maintain a physiologically appropriate alveolar surface tension, and further injury may result. An exogenous means of lowering surface tension should reduce the incidence and/or severity of ventilation injuries.

### Physiology of Acute Respiratory Distress Syndrome (ARDS)

Acute respiratory distress syndrome is associated with inflammation in the lungs, and can be caused by any number of different initial insults, including infection, aspiration, etc. During inflammation, there is increased permeability of the alveolar-capillary barrier, and liquid leaks out of the blood vessels. The liquid contains albumin, the most abundant plasma protein, as well as other plasma proteins (e.g., fibrinogen) that are present at lower concentrations. When enough liquid escapes from the vessels, liquid begins to collect in the alveoli, a condition known as alveolar edema. In flooded alveoli, the air-liquid interface forms a meniscus. As described by the Laplace relation, the liquid pressure in a flooded alveolus is less than the corresponding air pressure to a degree that is proportional to T at the meniscal interface. The additional liquid in the airspace effectively thickens the alveolar-capillary barrier, reducing the efficiency of gas exchange so that the patient becomes hypoxic.

The Laplace relation may be stated:

P_{LIQ}=P_{ALV}−2T/r_{M}. (1)

Here, P_{LIQ} refers to the liquid pressure on one side of the meniscus, P_{ALV} refers to the alveolar air pressure on the other side of the meniscus, T is the surface tension at the meniscal interface as described above, and r_{M} is the radius of the meniscus.

In alveolar edema, there may be regions of the lungs in which alveolar flooding is heterogeneous. Stated another way, aerated and liquid-flooded alveoli may be interspersed throughout the lungs. ‘Intervening’ septa, i.e., those located between adjacent aerated and flooded alveoli, are thus subjected to a relatively high air pressure, P_{ALV}, on one side and a relatively low liquid pressure, P_{LIQ} which is <P_{ALV}, on the other. The air-liquid pressure difference across the intervening septum, which equals the pressure difference across the meniscus of the flooded alveolus and is proportional to surface tension, causes the intervening septum to bow into the flooded alveolus. Thus, at any given lung inflation pressure, the intervening septum is extended beyond its normal length to a degree that is proportional to T, and is thereby subject to a stress concentration that is proportional to T.

### Ventilation-Induced Lung Injury (VILI) in ARDS

In ARDS, ventilation can be injurious. Due to the presence of heterogeneous flooding, even spontaneous breathing, during which each inhalation transiently increases T, can exacerbate stress concentrations in intervening septa, and may exacerbate the degree of injury/cause VILI. Further, patients with ARDS are often treated by mechanical ventilation, which assists gas exchange but can likewise exacerbate the degree of lung injury/cause VILI. Thus, mechanical ventilation can impede patient recovery. In particular, either type of ventilation distends portions of injured/edematous lungs to a greater degree than when the lungs are healthy, i.e. exacerbates the surface tension-dependent stress concentrations in intervening septa between aerated and flooded alveoli. When heterogeneous flooding is present in lungs in which the alveolar-capillary barrier is initially intact, ventilation injuriously increases permeability of the alveolar-capillary barrier to a degree that is proportional to the surface tension of the alveolar liquid in the region. As over-distension injury is surface tension-dependent, lowering surface tension of the liquid in the alveoli of an edematous lung should directly lessen ventilation injury.

Further, the heterogeneous alveolar flooding pattern is attributable to liquid being trapped in discrete alveoli by a ‘pressure barrier,’ i.e., the presence of a higher liquid pressure at the edge than in the center of flooded alveoli. The pressure barrier is proportional to surface tension at the air-liquid interface. Lowering surface tension can, by lowering the pressure barrier, facilitate liquid escape from flooded alveoli and redistribution, in a more homogeneous fashion, across neighboring alveoli. More homogenous (less heterogeneous) liquid distribution should reduce the number of septa subject to stress concentrations. Thus, lowering surface tension should also, by reducing flooding heterogeneity, indirectly reduce ventilation injury.

### Cardiogenic Pulmonary Edema (CPE)

In cardiogenic pulmonary edema, liquid entrance into the alveoli is driven not by abnormally elevated permeability of the alveolar-capillary barrier, but rather by abnormally elevated pulmonary capillary blood pressure secondary to left heart dysfunction. As barrier permeability is, at least initially, normal, plasma proteins should be trapped in the capillaries and plasma protein concentration in the alveolar edema liquid should be normal/low. However, quantitative analysis of alveolar liquid in CPE has demonstrated that protein concentration is elevated above normal in CPE, to the same degree as in ARDS. Further, in CPE, as in ARDS, there are regions of the lungs in which alveolar flooding is heterogeneous.

Without being bound by the correctness of any explanation or theory, either spontaneous breathing or mechanical ventilation is suspected to exacerbate stress concentrations in regions of heterogeneous alveolar flooding, thus causing VILI, i.e. injuring the alveolar-capillary barrier in those regions and leading to plasma protein entrance into the edema liquid. Regardless of the mechanism responsible for the elevated edema liquid plasma protein concentration in CPE patients, alveolar flooding pattern and edema liquid plasma protein concentration are similar between CPE and ARDS. In CPE, as in ARDS, lowering surface tension should, by either direct or indirect means, lessen ventilation injury of regions with heterogeneous alveolar flooding.

### Neonatal Respiratory Distress Syndrome (NRDS)

Lung surfactant is produced starting from the third trimester of gestation, and is critical to the ability of a baby to breathe unaided. Historically, many premature babies did not survive due to insufficient production of lung surfactant by immature lungs. Since the 1980's, tracheal instillation (administration) of exogenous animal surfactant has been a successful therapy that has enabled premature babies to live. However, there remains room for improvement in the clinical treatment of NRDS.

As newborn lungs are entirely filled with liquid prior to the first breath following birth, there are similarities between neonatal and edematous lungs. In NRDS, the low surfactant level causes T to be elevated. The high T impedes full inflation of the lungs following birth such that not all liquid is expelled. Thus in NRDS, as in ARDS, there can be interspersal of aerated and flooded alveoli. In another respect, NRDS is similar to CPE in that barrier permeability and alveolar liquid protein concentration should initially be normal/low. However, with stress concentrations present between aerated and flooded alveoli, mechanical ventilation or, in some cases, spontaneous breathing may cause VILI, thus increasing barrier permeability, and in turn, increasing alveolar liquid protein content. An important difference between NRDS and both ARDS and CPE is that in NRDS there is less surfactant present than in mature lungs.

In treatment of NRDS, exogenous surfactant therapy is already used to beneficially lower alveolar surface tension. However, lowering surface tension to a greater degree, or more uniformly throughout the lungs, should further lessen ventilation injury of regions with heterogeneous flooding.

### Surfactant Therapy

Surfactant therapy has been used as a method of lowering alveolar surface tension in some patients. As referred to herein, “surfactant therapy” refers to administration of an exogenous surfactant (e.g., a surfactant composition, mixture, suspension, component, or material) to the airways of the lungs. The administration may be carried out via instillation (e.g., as a liquid), aerosolization (e.g., nebulization), and/or lavage of any of a mixture, solution, suspension and/or composition. As used herein, “mixture,” “suspension,” “solution,” and “composition” are used in their art-recognized senses to refer to different modes of combining the constituent components. It is understood, however, that reference in this disclosure to one of “mixture,” “suspension,” “solution,” and/or “composition” also references all of these forms, i.e., “mixture,” “suspension,” “solution,” and “composition” such that a reference to “administration of a mixture,” for example, also inherently references administration of any of a mixture, suspension, solution or composition. As discussed above, surfactant therapy in NRDS has been highly successful, yet room remains for improvement. In ARDS, surfactant therapy has failed to reduce mortality in multiple randomized controlled clinical trials.

Possible reasons for the limited success or failure of surfactant therapy include heterogeneity of liquid and/or exogenous surfactant distribution throughout the lungs (e.g., due to problematic surfactant transport along the airways from the administration site, the trachea, to the target site, the alveoli). As noted above, the fact that high plasma protein concentrations are present in the alveolar liquid of premature neonates suggests that aeration, despite surfactant therapy, is sufficiently heterogeneous that stress concentrations are present and will be exacerbated by mechanical or spontaneous ventilation, resulting in injury to the alveolar-capillary barrier in NRDS. Accordingly, improved and/or adjuvant therapies for lowering surface tension and improving homogenous aeration are desired.

### Dye Components for Lowering Surface Tension

Certain dye compounds can be used to lower surface tension. Indeed, according to some embodiments of the present disclosure, any dye compound, component, solution or composition capable of lowering surface tension may be used as one of the surface tension-lowering components. Some non-limiting examples of suitable such dyes include select rhodamine dyes. Indeed, treatment with rhodamine dyes has also been explored as a method of treating edematous lungs. Such dyes include one or more anionic groups in addition to a positively charged imine group. Non-limiting examples of rhodamine dyes that may potentially be used to lower surface tension include rhodamine WT (RWT) and sulforhodamine B (SRB). SRB is approved as a food coloring in Japan and thought to be largely or substantially non-toxic. SRB has the following structure:

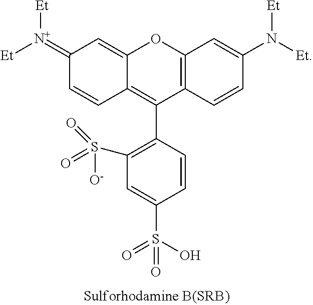 [SMILES: CCN(CC)c1ccc2c(-c3ccc(S(=O)(=O)O)cc3S(=O)(=O)[O-])c3ccc(=[N+](CC)CC)cc-3oc2c1]

Rhodamine WT and SRB are capable of lowering surface tension and promoting equitable redistribution of edema liquid among alveoli when administered to the lungs, as described in Perlman, U.S. Pat. No. 9,504,796, published Nov. 26, 2016 and titled “REDUCING VENTILATOR-INDUCED LUNG INJURY,” and in Perlman, U.S. Pat. No. 9,693,990, issued Jul. 4, 2017 and titled “USE OF RHODAMINE DYES TO REDUCE ALVEOLAR SURFACE TENSION,” the entire content of each of which is incorporated herein by reference. As a particular example, when injected into alveoli of isolated lungs in situ, SRB (at an alveolar liquid concentration of 1 nM-1,000 nM) has been shown to interact with albumin (at an alveolar liquid concentration of 3-12 w/v %) to lower surface tension and protect against ventilation injury of flooded alveoli, as described in Kharge, A B et al, “Sulforhodamine B interacts with albumin to lower surface tension and protect against ventilation injury of flooded alveoli,” J. Appl. Physiol. 2015, 118, 355-364; Nguyen, T L and Perlman, C E, “Sulforhodamine B Effect on Alveolar Surface Tension in Acute Respiratory Distress Syndrome Models,” Abstract submitted to the 2019 American Thoracic Society (ATS) International Conference, Oct. 30, 2018; Nguyen, T L and Perlman, C E, “Effects of sulforhodamine B and exogenous surfactant on alveolar surface tension in the acute respiratory distress syndrome,” Poster presented at the 2019 American Thoracic Society (ATS) International Conference, May 17-22, 2019; Nguyen, T L and Perlman, C E, “Sulforhodamine B and exogenous surfactant effects on alveolar surface tension under acute respiratory distress syndrome conditions,” J. Appl. Physiol. 2020, 129(6), 1505-1513; and Wu Y, Nguyen T, Perlman C E. Intravenous sulforhodamine B reduces alveolar surface tension, improves oxygenation and reduces ventilation injury in a respiratory distress model. Journal of Applied Physiology, 130: 1305-1316, 2021, the entire content of each of which is incorporated herein by reference.

The therapeutic efficiency of SRB has been found to vary for different disease state models. For example, alveolar administration of SRB was found to reduce T when T was increased by the presence of cell debris or secretory phospholipase A2 (sPLA₂), but not when T was increased by mucin or acid (e.g., as a model of gastric aspiration).

### Surface Tension Assessment Methods

The likely therapeutic efficiencies of various treatments can be evaluated by assessing their effects on alveolar surface tension. Surface tension can be assessed using a variety of suitable methods, as follows.

Method 1. Surface tension determination in the isolated adult rat lung. In an isolated (excised) adult rat lung, the alveolar liquid is labeled with a fluorescent dye verified not to alter surface tension. T is determined as follows at a curved region of the alveolar air-liquid interface (e.g., the meniscus in a flooded alveolus or the corner of an aerated alveolus). Alveolar air pressure is determined with a transducer at the trachea of the constantly-inflated lung. Alveolar liquid phase pressure is determined by servo-nulling pressure measurement. The three-dimensional interfacial radius of curvature is determined by confocal microscopy. The surface tension is calculated according to the Laplace relation.

Method 2. Ventilation ‘injury score’ in the isolated adult rat lung. An isolated adult rat lung is perfused with a physiologic solution (i.e. the solution is pumped through the lung vessels, including the capillaries) containing a low concentration of a fluorescent dye verified not to alter surface tension. A surface alveolus of the lung is micropunctured and a non-fluorescent test solution is injected. In experimental regions, a sufficiently large volume of liquid is injected to generate a pattern of heterogeneous alveolar flooding; in control regions, a sufficiently small volume of liquid is injected so that the liquid spontaneously clears from the region, leaving behind a micropunctured-but-aerated region. The region is imaged by confocal microscopy over a five minute baseline period at a constant transpulmonary pressure of 5 cm H₂O. Five ventilation cycles are supplied to the lung at 0.33 Hz with the equivalent of a positive end-expiratory pressure of 15 cm H₂O and with a tidal volume of 6 ml/kg body weight. The lung is then returned to a constant transpulmonary pressure of 5 cm H₂O and imaged for 10 additional minutes. Alveolar liquid fluorescence at all time points is normalized by capillary fluorescence.

At baseline, alveolar liquid fluorescence (in flooded alveoli of experimental regions or in the liquid lining layer of control, aerated regions) is low and constant in all regions. Following ventilation, alveolar liquid fluorescence remains unchanged in aerated regions but continually increases with time in heterogeneously flooded regions. This result indicates that in heterogeneously flooded, but not aerated, regions, ventilation injures the alveolar-capillary barrier, permitting fluorescence to pass from the vascular perfusate to the alveolar liquid, and the injury is sustained over time. The increase above baseline in normalized alveolar liquid fluorescence at the last time point of the experiment is used as an injury score. The injury score, which indicates the rate of increase of normalized fluorescence following ventilation, correlates with the surface tension of the test solution.

Method 3. Opening pressure of the immature fetal rat lung. To inflate the initially liquid-filled fetal lung for the first time, the pressure applied at the trachea must be sufficient to overcome a capillary force that is proportional to the surface tension of the liquid in the lung. Thus, following instillation of a test solution in the trachea of the immature fetal rat lung, the opening pressure of the lung is indicative of the surface tension of the test solution. At embryonic day 18 or 19 (term=day 22), a fetus is delivered from a pregnant rat by uterotomy. (The normalized phospholipid content of the fetal rat lung on embryonic day 19 is 65% of that at full term.) A test solution (4-5 μL) is placed in the tip of a cannula; the cannula is inserted into the trachea and fixed in place with a suture; and a column of water, behind an air-filled cylinder that is connected to the tracheal cannula, is used to raise tracheal pressure in steps (e.g., 10 cm H₂O steps). The opening pressure that causes air to flow into the lungs is recorded, and is proportional to the solution surface tension.

Method 4. Surface tension in a liquid drop. The surface tension in a drop of liquid (normal saline+31 μM fluorescein, which does not alter surface tension, for fluid visualization+test solutes) is determined using the same method as in the isolated adult rat lung (method #1, above). The liquid pressure in the drop is determined by servo-nulling pressure measurement; the interfacial radius of curvature is determined by confocal microscopy; and the air pressure is atmospheric. Surface tension is calculated according to the Laplace relation and found to be 72±2 mN/m for normal saline, as expected.

### Demonstration of Dye Treatment Administered via the Vasculature

The therapeutic efficiencies of clinical method analogues, including tracheal instillation, intravascular (IV) administration, etc. of various surface tension-lowering components can be tested in an animal following generation of model lung injury in the animal. For example, therapeutic IV SRB administration was tested in vivo in rats with model lung injury, as follows. All animals were handled in accord with a protocol approved by the Stevens Institute of Technology Institutional Animal Care and Use Committee. First, the model lung injury was generated by ventilating an anesthetized rat with an excessive tidal (breath) volume (V_{T}) of 42 ml/kg body weight and an end-expiratory pressure of zero for 15 min to physically injure the lungs. Then, modeling clinical treatment of a patient with lung injury, V_{T} was reduced to a protective level of 6 ml/kg, end-expiratory pressure was increased to a positive, protective level of 2 or 10 cm H₂O, and the rat continued to be supported by mechanical ventilation for 4 hrs. SRB was administered IV, targeting a plasma concentration of 10 nM, after the injury period and at the start of the support period, mimicking therapeutic administration after the development of respiratory distress but prior to supportive treatment by mechanical ventilation. Oxygenation was tracked during the 4-hr support period. At the end of the support period, the rat was sacrificed, the lungs were isolated, and alveolar T was determined in situ in the isolated lungs using Method 1, above. In lungs from rats sacrificed at the end of the 4-hr support period, T was determined in one aerated alveolus and one flooded alveolus.

When IV SRB was administered to rats according to the above-described protocol, it was found to (i) reduce surface tension specifically in flooded/injured alveoli, (ii) improve oxygenation, (iii) reduce VILI and (iv) reduce plasma markers of lung injury and inflammation. A detailed description of the protocol and associated experimental results can be found in Wu, Y et al., “Intravenous sulforhodamine B reduces alveolar surface tension, improves oxygenation and reduces ventilation injury in a respiratory distress model,” J. Appl. Physiol. 2021, 130, 1305-1316 (https://journals.physiology.org/doi/abs/10.1152/japplphysiol.00421.2020), the entire content of which is incorporated herein by reference. Given that IV SRB acts specifically in flooded/injured alveoli, which may not be ventilated and may be particularly difficult to reach with a therapy administered via the airways, a surface tension-lowering component (such as SRB or another substance) administered via the vascular system could be administered as a complement to a surface tension-lowering component administered via the airways.

### Complementary Administration of Surface Tension-Lowering Components

Embodiments of the present disclosure provide a method of treating a patient having edematous or liquid-flooded lungs (and/or elevated alveolar surface tension), by administering a surface tension-lowering component to the patient via the airways as well as administering a surface tension-lowering component via the vascular system (e.g., using IV injection). In some embodiments, the two administrations may be effected simultaneously, but the present disclosure is not limited to such simultaneous administration, and the co-administrations may occur at different times, which may or may not be near in time to each other and which may or may not overlap in time. The particular combination of treatment methods (routes) may be advantageous due to several complementary spatial and temporal aspects. In particular, because the treatments are applied via different routes, they are likely to reach different regions of the peripheral airspace of the lungs. For example, the component administered via the vascular system may perfuse throughout the alveolar tissues and thus reach portions of the lungs that are less accessible via the airways, thus ensuring that a larger fraction of alveoli are subjected to and can benefit from at least one of the treatment methods. Additionally, in some embodiments, the component administered via the vascular system may act on a different or faster timescale than the component administered via the airways. Thus, according to embodiments of the present disclosure, the complementary administration of the two therapeutics by different routes may more efficiently and effectively lower or normalize T compared to conventional methods.

According to aspects of embodiments of the present disclosure, a method of treating a patient having edematous or liquid-flooded lungs includes: administering a surface tension-lowering component to the patient via the patient's airways, and administering a surface tension-lowering component to the patient via the patient's vasculature.

The surface tension-lowering components provided to the airways and to the vasculature may be provided in therapeutically effective amounts. As referred to herein, therapeutically effective amounts of these components are those amounts that, directly or indirectly, minimize ventilation injury to an edematous or liquid-flooded lung by reducing the surface tension of alveolar liquid so that stress concentrations and/or alveolar flooding heterogeneity are reduced. Those having ordinary skill in the art are capable of determining such amounts according to the principles described herein. As understood by those having ordinary skill in the art, effective therapeutic dosing is dependent on the patient, and must be adjusted for various factors such as size (body weight), metabolism, and age. For example, a patient's plasma volume varies according to weight and hematocrit values, and the plasma volume may need to be included in calculations of a therapeutically effective amount, for example when a component is administered via the vasculature. Thus, as described herein, therapeutically effective amounts of the components may provide or result in concentrations of these substances in the alveolar liquid and/or blood plasma that are capable of reducing the surface tension of the alveolar liquid. In addition, effective therapeutic dosing may vary with respect to the methods and/or routes of administration, as well as additional materials within the components. Although examples presented herein may refer to certain materials, surfactants, etc., such examples are provided only as illustrations and do not limit the scope of embodiments of the present disclosure.

### Administration of Surface Tension-Lowering Component to Airways

The surface tension-lowering component may be administered to the patient's airways (e.g., via the trachea) using various suitable processes or treatments. In some embodiments, for example, the administering of the surface tension-lowering component via the airways may include tracheal instillation (e.g., administering the surface tension-lowering component as a liquid to the patient's trachea and/or bronchi) or tracheal aerosolization (e.g., nebulizing the surface tension-lowering component and allowing the particles to enter the patient's airway). In some embodiments, the administering may be accomplished through lavage (i.e., washing) of the airways. The surface tension-lowering component administered via the airways may include any suitable concentration of components and be administered in any suitable amount.

In some embodiments, the surface tension-lowering component administered via the airways may be a surfactant, or may include at least one surfactant (e.g., phospholipid-based surfactant). The surfactant may be described as being exogenous, in contrast to the endogenous surfactant produced within the patient's lungs. As surfactants remain at an air-liquid interface (e.g., do not distribute within a liquid phase), the surfactant is expected to spread along the interface and travel toward the alveoli. Any suitable surfactant or mixture of surfactants may be used

In some embodiments, for example, the surfactant may be a natural surfactant derived from animal sources (such as bovine or porcine lung surfactant). In some embodiments, the surfactant may include recombinant or synthetic components, including recombinant or synthetic SP-B and/or SP-C. In some embodiments, the exogenous surfactant may be a commercially available product. Non-limiting examples of such exogenous surfactants include SURVANTA® (Abbvie, Inc., North Chicago, Ill.), INFASURF® (ONY Biotech, Amherst, NY), and CUROSURF® (Chiesi Farmaceutici, S.p.A., Parma, Italy). In some embodiments, the exogenous surfactant may further include one or more salts or buffering agents, as understood to be suitable in the art.

In some embodiments, the surface tension-lowering component administered via the airways may be or include a diluted exogenous surfactant. For example, the surfactant may be diluted in water or a buffered solution (e.g., normal saline, Ringer's solution, physiologic saline solution, or any equivalent) such that its phospholipid concentration is less than those of the commercial surfactants listed above, but still concentrated enough to lower T in the lungs. In this case, the diluted exogenous surfactant (used as the surface tension-lowering component) may be used to lavage the airways.

In some embodiments, the surface tension-lowering component administered via the airways may be, or may include, a mixture of exogenous surfactant that is highly diluted to the point where it no longer effectively lowers T in the lungs on its own and a negatively charged solute (e.g., a plasma protein such as albumin and/or fibrinogen, or negatively charged dextran) that facilitates the highly diluted surfactant. Consequently, in the presence of the negatively charged solute, the highly diluted surfactant does lower T in the lungs. As an example, dilute 1 vol % SURVANTA® (a surfactant) in normal saline may not be sufficiently surface active, but 1-5 vol % SURVANTA® may be sufficiently surface active in the presence of 5 weight/volume % (w/v %) albumin (as a negatively charged solute). As another example, dilute 1 vol % SURVANTA® is also surface active in the presence of 5% fibrinogen or 5% negatively charged dextran (i.e. 5% dextran plus 10 μM NaOH, to impart a negative charge on the dextran). Here, (w/v %) is based on the weight (in grams) of the negatively charged solute and the volume (in tenths-of-liters) of liquid in which the negatively charged solute is dispersed. The exogenous surfactant may be diluted in water or a buffered solution. Those having ordinary skill in the art are capable of determining the concentration range of exogenous surfactant that is consistent with this embodiment. In this case, the mixture of highly diluted exogenous surfactant may be administered to the airways by instillation, aerosolization, and/or lavage. Further details regarding the use of plasma proteins are found in Wu at al., Lung ventilation injures areas with discrete alveolar flooding, in a surface tension-dependent fashion, J Appl Physiol, 2014, 117: 788-796, the entire contents of which are incorporated herein by reference.

In some embodiments, albumin may be present in the alveolar edema liquid within a suitable range (for example, 3-11 w/v %) to facilitate the highly diluted surfactant, and no additional albumin or other negatively charged solute may be needed or added with the highly diluted exogenous surfactant. Otherwise, in some embodiments, an additional negatively charged solute may be administered along with the highly diluted surfactant until the concentration of negatively charged solute in the alveolar edema liquid is within the desired range. In some embodiments, the negatively charged solute may be administered with the highly diluted surfactant to the airways (e.g., as a co-mixture, or in series), and in some embodiments, the negatively charged solute may be administered via the vasculature.

Surfactant protein C. SP-C is a 4.2 kilodalton (kD), 34 amino acid peptide. It has an N-terminal region of undefined conformation and an α-helix. Two cysteine residues in the N-terminal region are palmitoylated. When one or both palmitoyls is removed, the cysteine residues tend to form cross-bridges and the α-helix transforms into a β-sheet, such that the SP-C becomes denatured. Various forms of recombinant SP-C and synthetic SP-C (sSP-C) have been identified and/or tested as a component of synthetic surfactant, in which the role of the recombinant or synthetic SP-C would be to promote lipid adsorption. One such form is the unpalmitoylated sSP-Cff-ion lock (GIPFFPVHLKRLLIVVVVVELIVKVIVGALLMGL (SEQ ID NO:3)) which is disclosed in U.S. Patent Application Publication No. 2015/0125515, the entire content of which is hereby incorporated herein by reference. In this peptide, phenyalanine residues are substituted for the two cysteines, to avoid cross bridge formation and consequent SP-C aggregation in the absence of palmitoylation. Additionally, a glutamine with a negatively charged side chain and a lysine with a positively charged side chain are substituted within the α-helix region at residues 20 and 24, respectively. The oppositely charged side chains, located approximately one turn of the α-helix apart, are thought to attract one another and thus form an ‘ion lock’ that stabilizes the α-helix and prevents denaturation despite the lack of palm itoylation. Alternatively, also as disclosed in U.S. Patent Application Publication No. 2015/0125515, serine residues may be substituted for the two cysteins in the N-terminal region, to avoid cross bridge formation and aggregation in the absence of palmitoylation. And/or instead of using the ion lock to stabilize the α-helix, leucines may be substituted for valines in the α-helix region. Leucines, with longer side chains than valines, are an alternative means of helping to maintain α-helix integrity. When phenylalanines are substituted for the cysteines in the N-terminal region and leucines are used to stabilize the α-helix, the sSP-Cff-leuc peptide sequence is GIPFFPVHLKRLKLLLLLLLLILLLILGALLMGL (SEQ ID NO:2).

In the patent publication identified in the preceding paragraph it was reported that low concentrations of isolated SP-C or of sSP-C, in the presence of albumin, can lower surface tension in the lungs and thereby minimize mechanical ventilation injury to an edematous or liquid-flooded lung region. The following were tested: human SP-C isolated from pulmonary alveolar proteinosis patients, with albumin; sSP-Cff-ion lock, with albumin; sSP-Cff-ion lock-B, a variant of sSP-Cff-ion lock with a biotinylated N-terminal, with albumin; and sSP-Cff-leuc, with albumin. In the case of SP-C from pulmonary alveolar proteinosis patients, the SP-C should have been a mixture of peptides with two, one and zero attached palmitoyls, thus of SP-C with an intact α-helix and of denatured SP-C, and it is not known which form of SP-C was responsible for lowering surface tension. For all forms of SP-C and sSP-C, it is believed that, besides albumin, an alternative negatively charged solute, such as fibrinogen or negatively charged 70 kD dextran, would cooperate with low concentrations of the SP-C or sSP-C to lower surface tension in the lungs and thereby minimize ventilation injury to an edematous or liquid-flooded lung. Based on these findings, it is believed that a recombinant or synthetic SP-C, alone, could constitute a synthetic surface tension-lowering component that could achieve the aforesaid goal. The sequence listings for unpalmitoylated sSP-Cff-ion lock and sSP-Cff-leuc are presented in the attached sequence listing file.

In some embodiments, the surface tension-lowering component administered via the airways may be or include a SP-C that, when facilitated by a negatively charged solute (e.g., albumin, fibrinogen, or negatively charged dextran), may interact with native lung surfactant and lower T in the lungs. The SP-C may be natural, recombinant or synthetic. The recombinant or synthetic SP-C peptides that could be used include sSP-Cff ion lock; sSP-Cff ion lock-B; sSP-Cff-ion lock with biotin tags(s) in alternative locations; sSP-Cff-leuc; biotinylated sSP-Cff-leuc; sSP-Css ion lock (GIPSSPVHLKRLLIVVVVVELIVKVIVGALLMGL (SEQ ID NO:1)); sSP-Css ion lock-B; sSP-Css-ion lock with biotin tag(s) in alternative locations; sSP-Css-leuc; biotinylated sSP-Css-leuc; or alternative variations of natural human or animal SP-C. The sequence listing for sSP-Css-ion lock is presented in the attached sequence listing file

Suitable recombinant or synthetic SP-Cs are reasonably believed to include any of those identified in, e.g., Perlman, U.S. Patent Publication No. 2020/0046808, filed Aug. 15, 2019 and titled “DILUTE SURFACTANT OR ISOLATED SURFACTANT PROTEIN SOLUTION FOR THE REDUCTION OF SURFACE TENSION IN THE LUNG,” and in Perlman, U.S. Pat. No. 10,391,151, issued Aug. 27, 2019 and titled “DILUTE SURFACTANT OR ISOLATED SURFACTANT PROTEIN SOLUTION FOR THE REDUCTION OF SURFACE TENSION IN THE LUNG,” the entire content of each of which is incorporated herein by reference. The SP-C may be delivered in an aqueous vehicle or buffer. In some embodiments, as discussed above, albumin may be present in the alveolar edema liquid in a suitable range (for example, 3-11 w/v %) so that no additional negatively charged solute is needed. In some embodiments, additional negatively charged solute may be administered along with the SP-C in a manner similar to that described above (e.g., delivered to the airways within the aqueous vehicle used to administer the SP-C, or delivered via the vasculature).

As SP-C is hydrophobic, mixing SP-C with the aqueous buffer may be enhanced by inclusion of albumin in the aqueous buffer or by biotinylation of the SP-C. For example, in some embodiments, the surface tension-lowering component administered via the airways and including SP-C may further include albumin. In some embodiments, the SP-C may be biotinylated. In more detail, albumin has hydrophobic pockets, and may act as a carrier to solubilize the SP-C.

In practice, with human patients having an edematous or liquid-flooded lung and receiving mechanical ventilation, the volume of liquid of concern in the body (i.e., the volume of liquid in which either SP-C or a negatively charged solute, or both, is to be dispersed) is understood to be the sum of the edema liquid and blood plasma. Persons of ordinary skill in the art will be familiar with this principle and capable of calculating an estimated volume for a particular patient in need of receiving treatment, as well as calculating the therapeutically effective amount of SP-C or negatively charged solute necessary which will provide the concentrations discussed below.

The following discussion is intended to provide guidance without limiting the methods described and contemplated herein. Functional residual capacity (FRC), which is the air volume in the lung at the end of expiration, averages 2.3 liters (L) in adult humans. In pulmonary edema, permeability of the lung capillaries is elevated such that solutes, such as SP-C and a negatively charged solute, can pass between the alveolar edema liquid and the blood plasma. Therefore, the plasma volume, which averages 3 L, should be included in the calculation of the volume of concern. As recognized by persons of ordinary skill in the art, a particular human patient's weight and hematocrit values will aid in estimating the plasma volume.

Assuming that, in an adult human patient with pulmonary edema, somewhere between 5 and 80% of FRC were flooded with liquid, then the total volume of edema liquid would be 0.12-1.8 L (based on the average 2.3 L mentioned above) and the total volume of edema liquid plus blood plasma would be 3.1-4.8 L. This would be the total volume of the liquid of concern upon which to base further calculations of the range of amounts of SP-C and the negatively charged solute that would be required to be therapeutically effective at providing the concentrations discussed below. Calculations similar to these and based on FRC and blood volume can also be performed in applications dealing with treatment of premature babies with respiratory distress.

More particularly, therapeutically effective amounts of SP-C and the negatively charged solute are those amounts that, directly or indirectly, minimize mechanical ventilation injury to an edematous lung by reducing the surface tension of alveolar liquid so that stress concentrations and alveolar flooding heterogeneity are reduced. As discovered and described herein, therapeutically effective amounts of SP-C and the negatively charged solute are those amounts that provide the concentrations of these substances in the volume of liquid of concern as discussed hereinbelow because those concentrations of SP-C and the negatively charged solute reduce the surface tension of alveolar liquid.

From the results of testing based on the four methods described in the background above, it has been found that:
1. Dilute surfactant or an SP-C requires facilitation by a negatively charged solute. A dilute mixture containig 1 vol % SURVANTA® in normal saline is not surface active, but mixtures containing 1-5 vol % SURVANTA® are surface active when facilitated by inclusion of 5 vol % albumin and a mixture containing 1 vol % SURVANTA® is surface active when facilitated by inclusion of 5 vol % fibrinogen or of 5 vol % dextran plus 10 μM NaOH.

SURVANTA® contains 2.5% total phospholipids, which includes 1.1-1.6% DPPC, and <0.1% of SP-B and SP-C combined, with a concentration of SP-C that is up to 15 times that of SP-B. Thus 1% SURVANTA® contains 0.025% total phospholipids, ^{˜}0.01% DPPC, <0.001% SP-C and <<0.001% SP-B. The surface activity of mixtures containing DPPC, SP-B and SP-C, in concentrations comparable to those in 1 vol % SURVANTA®, was assessed in the absence and presence of 5 w/v % albumin and it was found that it is the SP-C in 1 vol % SURVANTA® that interacts with albumin or, presumably, with one of the other tested negatively charged solutes, to lower surface tension.

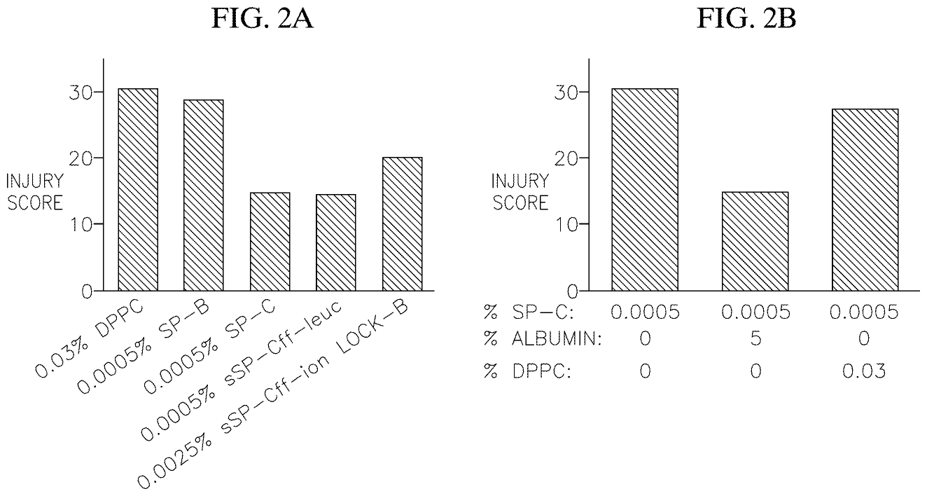

By ventilation injury assay (method #2), it was found that only mixtures containing SP-C or sSP-C but not those containing DPPC or SP-B, in conjunction with 5 w/v % albumin, lower injury score, and thus surface tension, as shown in FIG. 2. By the same method and in the absence of albumin, neither surfactant protein C alone nor the combination of SP-C and DPPC was surface active.

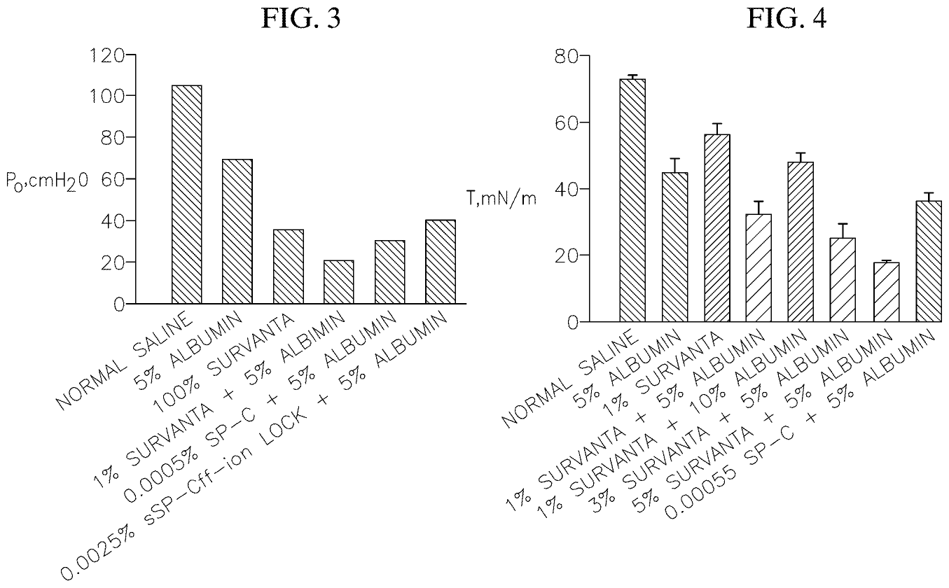

Likewise, in the fetal lung (method #3), SP-C or sSP-C plus 5 w/v % albumin lowered surface tension to the same degree as 1 vol % SURVANTA® plus 5 w/v % albumin, as shown in FIG. 3

The natural SP-C that was used in testing was isolated from the surfactant of pulmonary alveolar proteinosis patients, whose surfactant contains a mixture of normal SP-C with two attached palm itoyls as well as SP-C that is missing one or both palm itoyls. This mixture of normal and denatured SP-C species proved effective in experiments. For a therapeutic including natural SP-C, healthy SP-C (e.g., from an animal) would likely, but not necessarily, be preferable.

A mixture containing dilute surfactant or SP-C, where the SP-C may be natural, recombinant or synthetic, could be administered intratracheally by instillation, aerosolization or lavage in some embodiments. With sufficient albumin present in the alveolar liquid, dilute surfactant or SP-C solution could simply be administered in buffer (normal saline, Ringer's solution, physiologic saline solution, or equivalent). Without sufficient albumin present, a facilitating negatively charged solute (e.g., albumin, fibrinogen, negatively charged dextran or alternative negatively charged solute) could be added to the administered composition or administered intravascularly. The surfactant in the mixture of dilute surfactant may be SURVANTA® or another surfactant isolated from an animal that comprises SP-C.
2. A range of concentrations of albumin facilitate the surface activity of dilute SURVANTA® containing SP-C. Alternative negatively charged solutes (e.g., fibrinogen, negatively charged 70 kD dextran) also facilitate the surface activity of dilute SURVANTA® containing SP-C.

Albumin concentrations of 3-11 w/v % facilitate the surface activity of 1 vol % SURVANTA® in the adult rat lung. Alternatively, 5 w/v % fibrinogen or 5 w/v % negatively charged 70 kD dextran (negative charge imparted by inclusion of 10 μM NaOH) also facilitate 1 vol % SURVANTA®. In contrast, 5 w/v % neutral 70 kD dextran does not facilitate 1 vol % SURVANTA®. Thus osmotic pressure is not sufficient to facilitate 1 vol % SURVANTA®; a negatively charged solute is required. Control experiments have shown 10 μM NaOH alone, without dextran, has no effect on surface tension or lung injury in the absence or presence of SURVANTA®.

In vitro, albumin likewise facilitates the surface activity of SURVANTA®. However, the albumin concentration range that facilitates 1 vol % SURVANTA® does not extend to 10 vol % SURVANTA®.
3. At least a low concentration of lipids must be present in order for the combination of SP-C and albumin to lower surface tension. The combination of SP-C, or sSP-C, and albumin lowers surface tension in the adult rat lung with normal levels of native surfactant and in the immature fetal rat lung with reduced surfactant levels, as seen in FIGS. 2 and 3. Dilute 1-5 vol % SURVANTA® that contains SP-C and that contains only 0.03-0.13 w/v % total phospholipids (compared with 2.5 w/v % in undiluted SURVANTA®), in conjunction with 5 w/v % albumin lowers surface tension in vitro, as seen in FIG. 4. Thus the combination of SP-C and albumin is surface active in the presence of low lipid concentrations. Further, as also shown by the data in FIG. 4, dose-response experiments demonstrate that, in conjunction with 5 w/v % albumin, 5 vol % SURVANTA® lowers surface tension more than 1 vol % SURVANTA®.

However, the combination of SP-C and albumin in the absence of any lipids demonstrates only low surface activity (i.e., high surface tension) in vitro. The combination of SP-C and albumin appears to reduce surface tension by promoting the adsorption of surfactant lipids.

By extension of the above findings, it is expected that a concentration of from greater than about 2 w/v % to less than about 12 w/v % of a negatively charged solute (e.g., albumin, fibrinogen, and negatively charged 70 kD dextran), will facilitate the surface activity of a concentration of at least 0.001 vol % SURVANTA® or other surfactants isolated from animals; or of from about 0.000001 w/v % to about 1 w/v % SP-C, whether natural, recombinant or synthetic, in the presence of at least low levels of surfactant lipids, the relative proportions of which might be the same as or different from that in natural lung surfactant. This effect is reasonably expected regardless of whether the negatively charged solute is included in the composition delivered to the airways; or already present, for example, in edema liquid or blood plasma; or delivered via the vasculature. The recombinant or synthetic SP-C peptides that could be used include: sSP-Cff ion lock; sSP-Cff ion lock-B; sSP-Cff-ion lock with biotin tag(s) in alternative locations; sSP-Cff-leuc; biotinylated sSP-Cff-leuc; sSP-Css-ion lock; sSP-Css-ion lock-B; sSP-Css-ion lock with biotin tag(s) in alternative locations; sSP-Css-leuc; biotinylated sSP-Css-leuc; or alternative variations of natural human or animal SP-C, potentially including denatured forms of SP-C.

In some embodiments, the therapeutically effective concentration of SP-C (natural, recombinant or synthetic) in the liquid of concern may be, for example without limitation, from about 0.00001 w/v % to about 1 w/v %, or from about 0.0005 w/v % to about 1 w/v %, or from about 0.0001 w/v % to about 1 w/v %, or from about 0.005 to about 1 w/v %, or from about 0.0025 w/v % to about 1 w/v %, or from about 0.00001 w/v % to about 0.05 w/v %, or from about 0.0005 w/v % to about 0.05 w/v %, or from about 0.0001 w/v % to about 0.05 w/v %, or from about 0.005 to about 0.05 w/v %, or from about 0.0025 w/v % to about 0.05 w/v %, 0.00001 w/v % to about 0.01 w/v %, or from about 0.0005 w/v % to about 0.01 w/v %, or from about 0.0001 w/v % to about 0.01 w/v %, or from about 0.005 to about 0.01 w/v %, or from about 0.0025 w/v % to about 0.01 495 w/v %, or from about 0.00001 w/v % to about 0.1 w/v %, or from about 0.0005 w/v % to about 0.1 w/v %, or from about 0.0001 w/v % to about 0.1 w/v %, or from about 0.005 to about 0.1 w/v %, or from about 0.0025 w/v % to about 0.1 w/v %,

In some embodiments, the therapeutically effective concentration of the negatively charged solute in the liquid of concern may be, for example without limitation, from about 2.1 w/v % to about 11.9 w/v %, or from about 2.5 w/v % to about 11.9 w/v %, or from about 3 w/v % to about 11.9 w/v %, or from about 4 or from about 3 w/v % to about 11.9 w/v %, or from about 5 w/v % to about 11.9 w/v %, or from about 6 w/v % to about 11.9 w/v %, or from about 2.1 w/v % to about 11.5 w/v %, or from about 2.1 w/v % to about 11 w/v %, or from about 2.1 w/v % to about 10 w/v %, or from about 2.1 w/v % to about 9 w/v %, or from about 2.5 w/v % to about 11.5 w/v %.

Additional details regarding treatments with SP-C can be found in, e.g., Perlman, U.S. Patent Publication No. 2020/0046808, filed Aug. 15, 2019 and titled “DILUTE SURFACTANT OR ISOLATED SURFACTANT PROTEIN SOLUTION FOR THE REDUCTION OF SURFACE TENSION IN THE LUNG” in Perlman, U.S. Pat. No. 10,391,151, issued Aug. 27, 2019 and titled “DILUTE SURFACTANT OR ISOLATED SURFACTANT PROTEIN SOLUTION FOR THE REDUCTION OF SURFACE TENSION IN THE LUNG;” the entire content of each of which is incorporated herein by reference.

In some embodiments, the surface tension-lowering component administered via the airways may be or include an aqueous solution of a dye that, when facilitated by albumin, lowers T in the lungs. Non-limiting examples of suitable such dyes include rhodamine dyes, e.g., SRB and/or RWT. When albumin is present in the alveolar edema liquid within a suitable range (in this case, for example, 3-12 w/v %) to facilitate the dye (e.g., a rhodamine dye (or SRB and/or RWT)), no additional albumin or other negatively charged solute may be needed or added with the dye. Otherwise, in some embodiments, the aqueous solution administered to the airways may further include additional albumin or in some embodiments additional albumin may be administered via the vasculature; via either route the amount of additionally administered albumin would be selected to raise albumin concentration in the alveolar liquid to 3-11 w/v %.

### Administration of Surface Tension-Lowering Component to the Vasculature

In some embodiments, the surface tension-lowering component administered to the vasculature (e.g., IV) may include an aqueous solution of at least one T-lowering dye, for example, a rhodamine dye (e.g., in some embodiments, SRB and/or RWT) that, when facilitated by albumin, interacts with native lung surfactant and lowers T in the lungs. When albumin is present in the alveolar edema liquid within a suitable range (for example, 3-12 w/v %) to facilitate the dye (e.g., a rhodamine dye (or SRB and/or RWT)), no additional albumin or other negatively charged solute may be needed or added with the dye. Otherwise, in some embodiments, the aqueous solution may further include additional albumin to be administered to the vasculature, or in some embodiments may be administered via the airways. The surface tension-lowering component may be administered to the vasculature in any suitable form, without limitation. For example, the surface-tension lowering component may be administered to the vasculature in the form of a solution or mixture that includes the T-lowering dye, either by itself, or in solution or mixture with additional components. In some embodiments, for example, the surface tension-lowering component administered to the vasculature may include a solution or mixture that includes the T-lowering dye as well as one or more salts or buffering agents, as understood to be suitable in the art. For example, the one or more salts may include NaCl (saline), KCl, etc. The T-lowering dye solution or mixture may be provided in any therapeutically effective concentration in the blood plasma (which is about 3 L on average, as described above). In some embodiments, for example, when the surface tension-lowering component administered to the vasculature includes a rhodamine dye solution (e.g., SRB and/or RWT), the dye solution may be prepared so that the dye is provided at a target alveolar liquid concentration of about 1 nM to about 10,000 nM, about 1 nM to about 1,000 nM (1 μM), about 2 nM to about 800 nM, about 3 nM to about 600 nM, about 4 nM to about 500 nM, about 5 nM to about 400 nM, about 6 nM to about 300 nM, about 7 nM to about 200 nM, about 8 nM to about 100 nM, about 10 nM to about 80 nM, about 15 nM to about 60 nM, or about 20 nM to about 40 nM, where 1 nM is thought to be the minimum-effective alveolar concentration. As the concentration of dye (e.g., rhodamine dye) in the alveolar liquid may be equal to or less than that in the plasma, the dye may be correspondingly provided at a target plasma concentration higher than the above-described values, for example, 10%, 20%, 30%, 40%, 50%, 100%, 200%, 300%, 400%, 500%, 600%, 700%, 800%, 900% or 1,000% higher. However, embodiments of the present disclosure are not limited thereto.

It is noted that, at 4.2 kD, which is smaller than the 66 kD albumin that passes from capillary to alveolus in ARDS, CPE, and NRDS, SP-C peptide, like rhodamine dye, could be delivered intravascularly, potentially increasing either the homogeneity of the therapy throughout the lungs or the matching of the therapy to the edematous regions that require it.

In some embodiments, dilute surfactant or a mixture containing SP-C, where the SP-C may be natural, recombinant or synthetic, could be administered intravascularly, in the absence or presence of exogenous albumin or of an alternative negatively charged facilitating solute (e.g., fibrinogen or negatively charged dextran).

The dilute surfactant may be SURVANTA® or another surfactant isolated from an animal that comprises SP-C. When albumin is present in the alveolar edema liquid in a suitable amount to facilitate the dilute surfactant or the SP-C (e.g., an amount of 3-11 w/v %), no additional albumin or other negatively charged solute may be needed or added with the dilute surfactant or SP-C. In some embodiments, additional negatively charged solute may be administered along with the SP-C, for example via the airways and/or via the vasculature, to raise the concentration of the negatively charged solute to 3-11 w/v % in the alveolar liquid.

In some embodiments, solution or suspension of the SP-C in an aqueous vehicle may be facilitated by including albumin in that aqueous vehicle, or by biotinylation of the SP-C, as described above. Additional details regarding treatments with SP-C can be found in e.g., Perlman, U.S. Patent Publication No. 2020/0046808, filed Aug. 15, 2019 and titled “DILUTE SURFACTANT OR ISOLATED SURFACTANT PROTEIN SOLUTION FOR THE REDUCTION OF SURFACE TENSION IN THE LUNG,” and in Perlman, U.S. Pat. No. 10,391,151, issued Aug. 27, 2019 and titled “DILUTE SURFACTANT OR ISOLATED SURFACTANT PROTEIN SOLUTION FOR THE REDUCTION OF SURFACE TENSION IN THE LUNG,” the entire content of each of which is incorporated herein by reference.

### Timing of Administration Via Airways and Vasculature

In some embodiments, the administering of the surface tension-lowering component via the airways and the administering of the surface tension-lowering component via the vasculature may be started and/or carried out substantially simultaneously, though it is understood that the present disclosure is not limited to such. For example, in some embodiments, the administering of the surface tension-lowering component via the airways may be carried out prior to the administering of the surface tension-lowering component via the vasculature. And in some embodiments, administering of the surface tension-lowering component via the airways may be carried out after the administering of the surface tension-lowering component via the vasculature. The surface tension-lowering component delivered via the airways and the surface tension-lowering component delivered via the vasculature may comprise the same component, or can be different components. Furthermore, administration of either component may be carried out in multiple stages, repeated, as deemed necessary to support the patient or carried out over an extended period of time. In some embodiments, the administering of the surface tension-lowering component via the airways may be repeated at least once, and/or the administering of the surface tension-lowering component via the vasculature may be repeated at least once. However, it is not necessary that both components be administered in the same number of repeats. Accordingly, in some embodiments, administration of the surface tension-lower component via the airways may be repeated at least once, while administration of the surface tension-lowering component via the vasculature may not be repeated at all, and vice versa. Similarly, in some embodiments, administration of the surface tension-lowering component via the airways may be repeated any number of times, e.g., at least twice, while administration of the surface tension-lowering component via the vasculature may be repeated a different number of times, e.g., at least once, and vice versa. In some embodiments, the administering of the surface tension-lowering component via the airways may be carried out via continuous aerosolization and the administering of the surface tension-lowering component via the vasculature may be carried out via a continuous drip. When the administration of one, or both, surface tension-lowering component is repeated more than once and/or is continuous, the time periods during which the two surface tension-lowering components are administered may be coincident, may partially overlap or may not overlap.

In some embodiments, the method of treating the patient may further include mechanically ventilating the patient's lungs before, during and/or following the administering of the surface tension-lowering component via the airways, and/or before, during and/or following the administering of the surface tension-lowering component via the vasculature. In some embodiments, the patient is not mechanically ventilated.

The following examples are provided for illustrative purposes only, and do not limit the scope of the embodiments of the present disclosure.

### Examples and Experimental Data

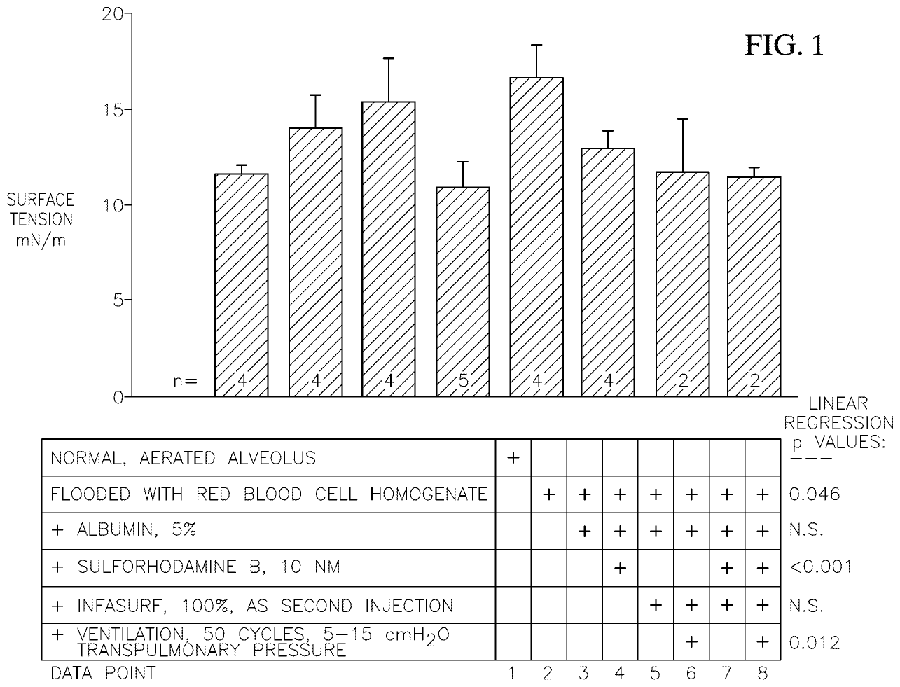

Alveoli of isolated rat lungs were injected (or flooded) in situ with various solutions or components and the effects on alveolar surface tension were compared. The accompanying FIG. 1 includes a composite bar chart comparing T values resulting from treatment with various solutions or components. Details, including the compositions and concentrations of any administered solutions or components, are shown below the x-axis of the chart. In more detail, the rat lungs were micropunctured, optionally flooded with red blood cell (RBC) homogenate alone, with albumin, or with albumin and SRB. Selected samples flooded with the RBC homogenate plus albumin were subsequently injected with a second injection of 100% INFASURF® (e.g., an exogenous surfactant). Half of these samples receiving the second injection were additionally subjected to 50 ventilation cycles at the noted pressures. Additional experimental details are described in the Methods section of Nguyen, T L and Perlman, C E, “Sulforhodamine B and exogenous surfactant effects on alveolar surface tension under acute respiratory distress syndrome conditions,” J. Appl. Physiol. 2020, 129(6), 1505-1513, the entire content of which is incorporated herein by reference.

With reference to the accompanying FIG. 1 and its chart, the first data point (bar) corresponds to normal, aerated alveoli in normal lungs, as a control. The red blood cell homogenate was added to the remaining data points to simulate epithelial damage and hemorrhaging of the alveolar capillaries, as may be present in some cases of ARDS. As shown in data points (bars) 2 and 3, the presence of the RBC homogenate resulted in an increase in T, without or with albumin. As albumin is normally present in edema liquid, data point (bar) 3 models the untreated clinical disease state.

As shown in data points (bars) 4, 7, and 8 of the aforesaid FIG. 1, alveolar T was immediately decreased/normalized, despite the presence of the RBC homogenate, when albumin and SRB were included. This effect was especially pronounced in comparison to data points 5 and 6, which show the effects of treatment with exogenous surfactant but not SRB (e.g., surfactant in place of SRB).

In the absence of SRB, administration of surfactant had no immediate effect but ventilation enabled the surfactant to lower surface tension (see data points 5 and 6 of FIG. 1). These data suggest that ventilation improves the efficacy of surfactant treatment.

Without ventilation, the combination of SRB and surfactant (see data point 7 of the chart) provided comparable results to SRB alone (data point 4) and improved results compared to surfactant alone (see data point 5 of the chart). The data suggest that SRB treatment acts faster than exogenous surfactant and might provide a dynamic advantage over surfactant. Thus SRB (or an analogous T-lowering dye or other substance) might be an effective complement or alternative to surfactant treatment, particularly when administered via the vascular system.

While certain exemplary embodiments of the present disclosure have been illustrated and described, those of ordinary skill in the art will recognize that various changes and modifications can be made to the described embodiments without departing from the spirit and scope of the present disclosure, and equivalents thereof, as defined in the claims that follow this description. For example, although certain components may have been described in the singular, i.e., “an” alveolus, “a” surfactant, and the like, one or more of these components in any combination can be used according to the present disclosure.

Also, although certain embodiments have been described as “comprising” or “including” the specified components, embodiments “consisting essentially of” or “consisting of” the listed components are also within the scope of this disclosure. For example, while embodiments of the present disclosure are described as comprising administration of an exogenous surfactant and a dye, embodiments consisting essentially of or consisting of these items are also within the scope of this disclosure. Accordingly, a therapeutic treatment may consist of administration of an exogenous surfactant and a dye, or may consist essentially of an exogenous surfactant and a dye. In this context, “consisting essentially of” means that any additional components or actions will not materially affect the performance of the treatment.

As used herein, unless otherwise expressly specified, all numbers such as those expressing values, ranges, amounts or percentages may be read as if prefaced by the word “about,” even if the term does not expressly appear. Further, the word “about” is used as a term of approximation, and not as a term of degree, and reflects the penumbra of variation associated with measurement, significant figures, and interchangeability, all as understood by a person having ordinary skill in the art to which this disclosure pertains. Any numerical range recited herein is intended to include all sub-ranges subsumed therein. Plural encompasses singular and vice versa. For example, while the present disclosure may describe “an” alveolus or “a” surfactant, multiple alveoli or a mixture of surfactants can be used. When ranges are given, any endpoints of those ranges and/or numbers within those ranges can be combined within the scope of the present disclosure. The terms “including” and like terms mean “including but not limited to,” unless specified to the contrary.

Notwithstanding that the numerical ranges and parameters set forth herein may be approximations, numerical values set forth in the Examples are reported as precisely as is practical. Any numerical value, however, inherently contains certain errors necessarily resulting from the standard variation found in their respective testing measurements. The word “comprising” and variations thereof as used in this description and in the claims do not limit the disclosure to exclude any variants or additions.

A detailed description of directly administering SRB to surface alveoli in the absence or presence of other substances modeling disease conditions can be found in Nguyen, T L and Perlman, C E, “Sulforhodamine B Effect on Alveolar Surface Tension in Acute Respiratory Distress Syndrome Models,” Abstract submitted to the 2019 American Thoracic Society (ATS) International Conference, Oct. 30, 2018; Nguyen, T L and Perlman, C E, “Effects of sulforhodamine B and exogenous surfactant on alveolar surface tension in the acute respiratory distress syndrome,” Poster presented at the 2019 American Thoracic Society (ATS) International Conference, May 17-22, 2019; and Nguyen, T L and Perlman, C E, “Sulforhodamine B and exogenous surfactant effects on alveolar surface tension under acute respiratory distress syndrome conditions,” J. Appl. Physiol. 2020, 129(6), 1505-1513, all of which have been incorporated by reference herein. It should be noted that the terms “solution,” “composition,” “mixture” and “suspension” are at times used interchangeably throughout the specification.

A detailed description of administering SRB and albumin to surface alveoli can be found in Kharge, A B et al, “Sulforhodamine B interacts with albumin to lower surface tension and protect against ventilation injury of flooded alveoli,” J. Appl. Physiol. 2015, 118, 355-364, which has been incorporated by reference herein.

A detailed description of NRDS and ARDS disease states and the beneficial effects of lowering surface tension via SP-C administration can be found in U.S. Patent Publication. No. 2020/0046808 and in U.S. Pat. No. 10,391,151, both of which have been incorporated by reference herein.

A detailed description of methods of using a rhodamine dye to reduce surface tension in lungs can be found in U.S. Pat. No. 9,504,796 and in U.S. Pat. No. 9,693,990, both of which have been incorporated by reference herein.

A detailed description of intravenous administration of SRB to lung-injured rats can be found in Wu, Y et al., “Intravenous sulforhodamine B reduces alveolar surface tension, improves oxygenation and reduces ventilation injury in a respiratory distress model,” J. Appl. Physiol. 2021, 130, 1305-1316 https://journals.physiology.org/doi/abs/10.1152/japplphysiol.00421.2020), which has been incorporated by reference herein.

1-43. (canceled)

44. A method of treating a patient undergoing respiratory distress and/or with excess liquid in the airspace of the lungs, the method comprising:
(i) administering a first surface tension-lowering component to the patient via the patient's airways; and
(ii) administering a second surface tension-lowering component to the patient via the patient's vascular system, the second surface tension-lowering component being administered substantially simultaneously with the administration of the first surface tension-lowering component and/or within a therapeutically effective time interval thereof.

45. The method of claim 44, wherein step (i) is performed over a first time period and step (ii) is performed over a second time period, which overlaps the first time period.

46. The method of claim 44, wherein step (i) is repeated more than once or continuously.

47. The method of claim 44, wherein step (ii) is repeated more than once or continuously.

48. The method of claim 44, wherein the second surface tension-lowering component is administered via a continuous infusion or drip or one or more injections.

49. The method of claim 44, wherein the first surface tension lowering component is administered via tracheal instillation, aerosolization or lavaging.

50. The method of claim 44, wherein the first surface tension-lowering component and/or the second surface tension-lowering component comprises at least one exogenous surfactant, or at least one surfactant protein C, or at least one rhodamine dye.

51. The method of claim 44, wherein the first surface tension-lowering component comprises at least one exogeneous surfactant and the second surface tension-lowering component comprises at least one rhodamine dye selected from SRB and/or RVVT.

52. The method of claim 44, further comprising the step of adding a negatively-charged solute to the first surface tension-lowering component and/or to the second surface tension-lowering component.

53. The method of claim 52, wherein the negatively-charged solute is added to the first surface tension-lowering component only.

54. The method of claim 52, wherein the negatively-charged solute is added to the second surface tension-lowering component only.

55. The method of claim 52, wherein the negatively-charged solute is added to both the first surface tension-lowering component and the second surface tension-lowering component.

56. The method of claim 52, wherein at least some of the at least one negatively-charged solute is administered via the patient's vasculature.

57. The method of claim 52, wherein the negatively-charged solute is selected from albumin, fibrinogen, and negatively-charged dextran.

58. The method of claim 57, wherein the first surface tension-lowering component and/or the second surface tension-lowering component comprises a surfactant protein C, and wherein the negatively-charged solute comprises albumin.

59. The method of claim 44, wherein the patient is undergoing respiratory distress due to NRDS or ARDS, or the patient has cardiogenic pulmonary edema.

60. The method of claim 50, wherein the at least one surfactant protein C or the at least one exogenous surfactant is highly diluted.

61. The method of claim 44, wherein the patient's lungs are mechanically ventilated before, during or after the administering of the first surface tension-lowering component.
